(19) United States
(12) Patent Application Publication
(10) Pub. No.: US 2022/0219015 A1
(43) Pub. Date: Jul. 14, 2022

(54) METHOD OF TREATMENT BY RADIOTHERAPY

(71) Applicant: James Chinan Chen, Rockville, MD (US)

(72) Inventor: James Chinan Chen, Rockville, MD (US)

(21) Appl. No.: 17/146,111

(22) Filed: Jan. 11, 2021

(51) Int. Cl. A61N 5/10; A61K 33/00; A61K 45/06

(52) U.S. Cl. CPC A61N 5/1049; A61K 33/00; A61N 2005/1052; A61N 2005/109; A61N 2005/1055; A61K 45/06

(57) ABSTRACT

Provided is a method for treating a target tissue in a subject by using a particle beam, or further in combination with a reactant. The method includes irradiating the target tissue with the particle beam by scanning under a controlled path in the target tissues.

### BACKGROUND

### 1. Technical Field

The present disclosure relates to methods for treating biological tissues based on radiotherapy, e.g., treating tissues with aberrant growth, cancerous tissues, or harmful tissues with metastatic potentials in a subject. The methods involve use of an irradiation device and a reactant to provide an efficient and precise treatment of the biological tissues.

### 2. Description of Related Art

Normally, cells of an organism are under scrutinous control in a cell cycle involving stages such as cell division, cell proliferation and cell death. When cells no longer respond to the signals that control cellular growth and death, they divide more rapidly than others and can evade from programmed cell death, resulting in cancer. In the late stages of cancer, cells break through normal tissue boundaries and metastasize to new sites in the body. Currently, cancer is the leading causes of death worldwide, accounting for 9.5 million deaths in 2018. In 2018 alone, a total of 18 million new cancer cases were diagnosed.

Cancers are treated in a number of ways including surgery, chemotherapy, radiation therapy, hormonal therapy, targeted therapy, cellular therapy and palliative care. Among these, particle therapy is one type of external beam radiation therapy where beams of energetic neutrons, protons, or other heavier positive ions such as carbon ions are used to irradiate the tissues to be treated.

Proton therapy, with a narrow range of depth of dose deposition and minimal entry, exit or scattered radiation dose, has emerged as the most-rapidly expanding hydrotherapy approach. The physical properties of charged particles, which can deposit energy far more selectively than photons through the inverted depth-dose profile described by the Bragg curve, enable the delivery of very high-dose gradients close to tissues at risk, confining the high-dose area to the target tumor tissues. Proton therapy thereby allows improved tumor treatment in the context of focusing the energy on the cancer cells at different depths in the body that reduces the risk of normal tissue damage.

However, despite the depth dose characteristic possessed by protons, there remains many challenges in effective delivery of the prescribed dose into the target tissue precisely while at the same time sparing as much as possible the neighboring healthy tissues and vital organs, considering the fact that a tumor is a mass taking up a 3-dimensional space and is often irregular in shape. This is true when the tissues to be treated involve a large size tumor, e.g., aggressive and metastatic cancer tissues that have spread, which seat deep inside the body or near delicate organs such as spinal cord and eyes. Hence, there remains a need in the art for means to control and deliver the dose precisely to the target tissue.

### SUMMARY

The present disclosure relates to a method for treating a target tissue in a subject in need thereof by using a particle beam. The method of the present disclosure comprises determining an isocenter of the target tissue and irradiating the target tissue with the isocenter as an initiation point by using a particle beam scanning under a controlled path. In at least one embodiment, the isocenter of the target tissue is determined with image analysis of a dimension and a location of the target tissue. In some embodiments, the image analysis is carried out with computed tomography (CT) scan, cone beam computed tomography (CBCT) scan, in-room CT scan, magnetic resonance imaging (MRI) scan, orthogonal X-ray, positron emission tomography (PET) scan, or any combination thereof.

In at least one embodiment of the present disclosure, the particle beam is a charged particle beam or an uncharged particle beam. In some embodiments, the charged particle beam is a proton beam, a carbon ion beam, an electron beam or any combination thereof. In some embodiments, the uncharged particle beam is a neutron beam. In some embodiments, the charged particle beam is a proton beam.

In at least one embodiment of the present disclosure, the particle beam irradiates the target tissue in a spot scanning manner, a uniform scanning manner, a fast scanning manner, a scatter manner, or any combination thereof.

In at least one embodiment of the present disclosure, the target tissue is defined by three coordinates, X, Y and Z, according to X, Y and Z directions, respectively, wherein the Z direction corresponds to a direction of the particle beam, and the X and Y directions are perpendicular to the Z direction.

In at least one embodiment, the particle beam irradiates the target tissue by scanning under a controlled path that forms a spiral pattern on the two-dimensional plane defined by the X and Y coordinates with a first fixed Z coordinate. In some embodiments, the scanning is repeated with the isocenter of the target tissue as the initiation point at another two-dimensional plane defined by the X and Y coordinates with a second fixed Z coordinate. In at least one embodiment, the Z coordinate of the irradiation scanning path is controlled by modulating the energy of the particle beam.

In at least one embodiment of the present disclosure, the isocenter of the target tissue is calibrated with one or more positioning points prescribed around the isocenter before initiating the scanning of each of the two-dimensional plane of the target tissue. In some embodiments, the lines connecting the positioning points and crossing to each other define the isocenter for calibration.

In at least one embodiment of the present disclosure, the method further comprising administering to the subject a first reactant comprising at least one of ¹⁰B and ¹¹B which reacts with at least one proton from the particle beam to release at least one of α particle and γ ray, and the α particle or the γ ray reacts with the target tissue. In some embodiments, the first reactant is selected from the group consisting of (L)-4-dihydroxy-borylphenylalanine (BPA), sodium mercaptoun decahydro-closo-dodecaborate (BSH), a carbohydrate derivative of BSH, a sodium salt of closo-B₁₀H₁₀²⁻ (GB-10), β-5-o-carboranyl-2V-deoxyuridine (D-CDU), 3-(dihydroxypropyl-carboranyl-pentyl) thymidine derivative (N5-20H), boron-containing porphyrin (H₂DCP), dequalinium derivative (DEQ-B), a derivative of trimethoxyindole, aziridine, a derivative of acridine, phenanthridine, carboranyl polyamine, a Pt(II)-amine complex, dibenzimidazole, tribenzimidazole, a glucose molecule, a mannose molecule, a ribose molecule, a fucose molecule, a galactose molecule, a maltose molecule, a lactose molecule, phosphate, phosphonate, phenylurea, thiourea, nitroimidazole, amine, benzamide, isocyanate, nicotinamide, and azulene.

In at least one embodiment of the present disclosure, the method further comprises a second reactant comprising a first composite and a second composite, wherein the first composite is provided for reacting with at least one proton from the particle beam and releasing at least one neutron in the target tissue, and the second composite is provided for reacting with the at least one neutron to release at least one of α particle or γ ray that reacts with the target tissue. In some embodiments, the first composite contains one selected from the group consisting of ⁷Li, ⁹Be and a combination thereof and the second composite contains ¹⁰B.

In at least one embodiment of the present disclosure, the method for treating a target tissue in a subject by using a particle beam further comprises implementing a treatment planning system (TPS) comprising a computer and a data storage device for storing the information for use in scanning the target tissue. In some embodiments, the stored information is retrieved for subsequent treatments.

In some embodiments, the method further comprises administering at least an additional therapy. For example, the at least one additional therapy may comprise chemotherapy, immunotherapy, surgery, radiotherapy, biotherapy or any combination thereof. In at least one embodiment, the immunotherapy may trigger death of cancer cells induced by CD8⁺ T cells. In some embodiments, the immunotherapy relates to administration of an immune checkpoint inhibitor. For example, enhanced ICOS-stimulated Th17 cells and/or an at least one additional therapeutic agent are administered intravenously, intraperitoneally, intratracheally, intratumorally, intramuscularly, endoscopically, intralesionally, percutaneously, subcutaneously, regionally, or by direct injection or perfusion.

In some embodiments, the immunotherapy comprises adoptive transfer of a T cell population. In some embodiments, the immunotherapy is treatment with an immune checkpoint inhibitor, a cytokine, or an immune modulator. For example, the immune checkpoint inhibitor is a PD-1 inhibitor or a CTLA-4 inhibitor. In some embodiments, the PD-1 inhibitor is nivolumab, pembrolizumab or the like.

In some embodiments of immunotherapy, the cancer cell or tumor cell may bear a marker that is amenable for targeting, i.e., not present on the majority of other non-cancerous cells. Many tumor markers exist and any of these may be suitable for targeting in the context of the present embodiments. Common tumor markers include CD20, carcinoembryonic antigen, tyrosinase (p97), gp68, TAG-72, HMFG, sialyl Lewis antigen, MucA, MucB, placental alkaline phosphatase (PLAP), laminin receptor, erb B, and p155. Alternative embodiments of immunotherapy are to combine anticancer effects with immune stimulatory effects. Immune stimulating molecules also exist that include: cytokines (such as IL-2, IL-4, IL-12, granulocyte-macrophage colony-stimulating factor (GM-CSF) and gamma-IFN), chemokines (such as macrophage inflammatory protein 1 (MIP-1), monocyte chemoattractant protein 1 (MCP-1) and IL-8), and growth factors (such as FLT3 ligand).

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the proton therapy described herein.

In some embodiments, the immunotherapy may be an immune checkpoint inhibitor. Immune checkpoint inhibitors are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In some embodiments, the immune checkpoint inhibitors target the PD-1 axis and/or CTLA-4.

The immune checkpoint inhibitors may be drugs such as small molecules, recombinant forms of ligands or receptors, or antibodies such as human antibodies (e.g., International Patent Publication WO 2015/016718; Pardoll 2012). Known inhibitors of the immune checkpoint proteins or analogs thereof may be used, e.g., chimerized or humanized, or human forms of antibodies may be used.

In some embodiments, the immunotherapy is administered after irradiating a target tissue in a subject with a particle beam. The immunotherapy targets the remaining cancer cells that have been broken up into smaller masses or small populations of cancer cells and thus clears up the cancer cells, thereby providing a comprehensive treatment for cancers and/or tumors.

These and others of this disclosure will become apparent from the following detailed description of the embodiments of the disclosure. Both the foregoing general descriptions and the following detailed descriptions are exemplary and explanatory only and are not intended to limit the scope of the present disclosure.

The accompanying drawings are included to provide a further understanding of the disclosure and are incorporated in and constitute a part of this specification, illustrate several embodiments of this disclosure, and together with the description serve to explain the disclosure.

### BRIEF DESCRIPTION OF THE DRAWINGS

The present disclosure will become more readily appreciated by reference to the following descriptions in conjunction with the accompanying drawings, wherein:

FIG. 1 indicates the spatial relation between the isocenter determined from the image analysis of the target tissue and the positioning points used for the calibration;

FIG. 2 shows the target tissue to be treated (shown as the dotted irregular region) with the determined isocenter and the positioning points;

FIG. 3 shows the spiral pattern of the controlled path of the particle beam in scanning the target tissue; and

FIG. 4 shows the X, Y and Z coordinates defining the target tissue to be scanned with the particle beam, where the Z coordinate corresponds to the beam direction while the X and Y coordinates correspond to the directions perpendicular to the Z direction.

### DETAILED DESCRIPTIONS

One or more embodiments of the present disclosure will now be described in detail with reference to the attached figures. This disclosure is not limited thereto but only by the claims. The drawings described are only schematic and are non-limiting. In the drawings, the size of some of the elements may be exaggerated and not drawn on scale for illustrative purposes. The dimensions and the relative dimensions do not necessarily correspond to actual reductions to practice of this disclosure. Those skilled in the art can recognize numerous variations and modifications of this disclosure that are encompassed by its scope. Accordingly, the description of embodiments should not be deemed to limit the scope of the present disclosure.

Furthermore, the terms first, second and the like in the description and in the claims are used for distinguishing between similar elements and not necessarily for describing a sequential or chronological order. It is to be understood that the terms so used are interchangeable under appropriate circumstances and that the embodiments of the disclosure described herein are capable of operation in other sequences than described or illustrated herein. All terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the detailed descriptions of the present disclosure. Thus, the terms used herein have to be defined based on the meaning of the terms together with the descriptions throughout the specification.

Also, when a part “includes” or “comprises” a component or a step, unless there is a particular description contrary thereto, the part can further include other components or other steps, not excluding the others.

It is further noted that, as used in this disclosure, the singular forms “a,” “an,” and “the” include plural referents unless expressly and unequivocally limited to one referent. The term “or” is used interchangeably with the term “and/or” unless the context clearly indicates otherwise.

The terms “subject,” “patient” and “individual” are used interchangeably herein and refer to a warm-blooded animal such as a mammal that is afflicted with, or suspected of having, at risk for or being pre-disposed to, or being screened for cancer, e.g., actual or suspected cancer. These terms include, but are not limited to, domestic animals, sports animals, primates and humans. For example, the terms refer to a human.

Particle therapy may be less harmful than other radiotherapy because it results in a lower total radiation energy, termed integral dose, being deposited in a patient for a given tumor dose in relation to other radiotherapy such as photon therapy. The integral dose reduction is significant because it reduces the probability of stochastic effects, i.e., patients developing secondary malignant neoplasms following irradiation of non-tumor tissues. In addition, charged particle therapy may also provide reduced radiation dose to healthy tissues sufficiently such that deterministic effects (i.e., complications whose magnitude is related to the radiation dose delivered) may be reduced relative to other radiotherapy. Examples of deterministic effects are skin erythema and xerostomia. The reduction in deterministic effects has been demonstrated in multiple studies, in which tumor dose conformity has been shown to be comparable to that of photon therapy, but healthy tissues spare for charged particle therapy such as proton therapy.

In this context, the prescribed radiation dose needs to be effectively delivered within the volume of the target tissue defined by radiotherapist, while at the same time sparing as much as possible the neighboring healthy tissues and vital organs. This is referred to as the “conformation” of the dose delivered to the target tissue. In order to reach a suitable conformation, a predefined radiation dose is calculated in order to reach a clinically useful dose distribution that conforms, as far as possible, the shape of the target tissue and contemporaneously spares the contiguous healthy tissues.

Therefore, a number of different beams spreading methods or systems have been developed as part of the proton therapy system to accurately and safely spread the proton beam over the prescribed treatment area, while minimizing the dose delivered to the normal tissue. A system of scatters, range compensators or range modulators provides the simplest way to spread a proton beam into a uniform field covering the required area. However, this passive beam spreading technique has drawbacks that cause beam losses and energy degradation of the therapeutic beam.

Another method used for beam spreading is termed as an “active” method and involves use of magnets. Magnetic beam scanning allows one to spread a proton beam over the desired radiation field area, improving beam utilization and conformity to the target area. Among these, one active beam spreading method involving a general pencil beam scanning system irradiates each voxel of the treatment volume independently to the prescribed dose, which requires simultaneous control of the beam position, shape, intensity, and energy. On the other hand, wobbling is a simpler version of an active beam spreading system that scans a constant intensity beam over a circular pattern many times to create a uniform radiation field. Uniform scanning systems generalize the wobbling approach to include arbitrary periodic scan patterns capable of generating uniform dose distributions. Wobbling can have multiple concentric circular trajectories, and the resulting pattern has point symmetry around the origin of the coordinate system.

In the circular wobbling method, the spiraling beam oscillates at constant frequency ω in the X and Y directions, while the oscillation amplitude changes with time to keep the particle density constant over the irradiated area:

X(t)=R₀\sqrt{ }t/τ cos(ω,t) (Formula Ia);

Y(t)=R₀\sqrt{ }t/τ sin(ω,t) (Formula Ib),

where τ is the period of the wobbling and R₀ is the maximum amplitude of the circular trajectory.

However, such beam spreading with wobbling leads to waste of radiation dose because of the vibration and wobbling frequency in the scanning path. In addition, there is no shielding design in the nozzle while wobbling, and when the particle beam irradiates normal cells, which will happen inevitably due to the irregular shape of tumor mass, the normal cells will be harmed and destroyed.

On top of the beam spreading mechanism, the present disclosure provides a method for scanning the target tissue with particle beams. In at least one embodiment, the target tissue is scanned in a spiral scan pattern initiating from the predetermined isocenter of the target tissue, with or without wobbling of the particle beam. The tissue scanning path in a spiral pattern can be made periodic and easily commissioned for large field sizes by simply extending the scan to add more spirals. The spiral scan path as disclosed herein provides a more uniform scan than zig-zag or linear scan patterns.

In at least one embodiment, the present disclosure comprises blocking the particle beam to avoid irradiation of normal cells by a shielding plate. When the scanning particle beam reaches the harmful cells, the baffle opens to allow irradiation of the particle beam for treatment, thereby destroying the harmful cells.

In at least one embodiment of the present disclosure, the spiral scanning path depends only on t (time), with the formulae for the X and Y directions as follows:

X(t)=R₀\sqrt{ }t/τ cos(t) (Formula IIa);

Y(t)=R₀\sqrt{ }t/τ sin(t) (Formula IIb),

where τ is the period of the spiraling scan pattern and R₀ is the maximum amplitude of the spiral.

Regardless of the beam spreading system used, the present disclosure provides a method for treating a target tissue in a subject using particle beams by first determining the isocenter of the target tissue, through prior image analysis of the target tissue, and then initiating the scanning of the particle beam starting from the isocenter of the target tissue. By starting from the isocenter, it is sure that the first spot to be scanned is the target tissue containing the cancerous cells that are aimed to be removed with the irradiation.

The image analysis of the target tissue to determine the isocenter can be done by any conventional techniques such as computed tomography (CT) scan, magnetic resonance imaging (MRI), digital X-ray or positron emission tomography (PET), cone beam computed tomography (CBCT), in-room CT, or by an orthogonal X-ray.

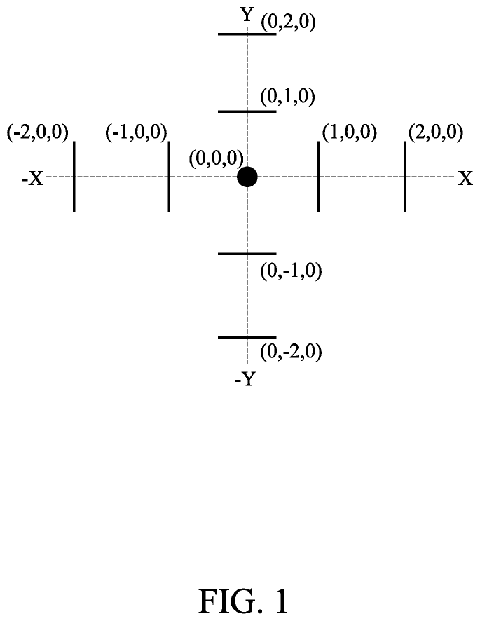

After the isocenter is determined, more positioning points can be assigned so as to facilitate the calibration of the isocenter to be targeted by the particle beam for each scanning. For example, as shown in FIG. 1, nine positioning points are assigned after image analysis of the target tissue with the isocenter located in the center having a coordinate (0, 0, 0), and 4 positioning points each to the top, bottom, left and right of the isocenter and all at equal unit distance to the isocenter, with coordinates (0, 1, 0), (0, −1, 0), (−1, 0, 0) and (1, 0, 0), respectively, and another 4 additional positioning points at the double unit distance further away and still all at equal distance from the isocenter, with coordinates (0, 2, 0), (0, −2, 0), (−2, 0, 0) and (2, 0, 0), respectively. The isocenter, therefore, is where the lines connecting the positioning points and the isocenter cross each other.

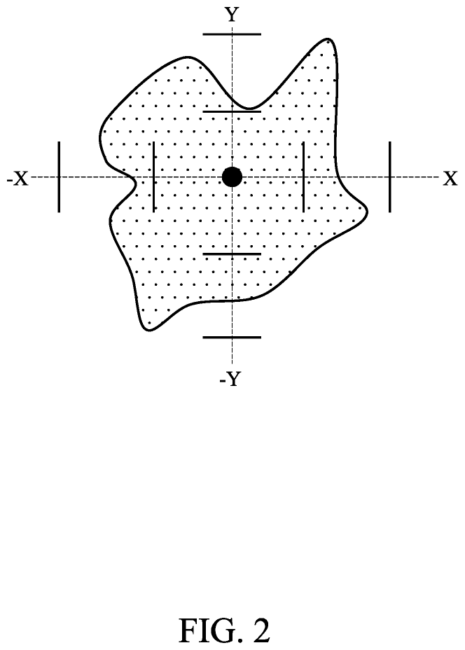

FIG. 2 shows the shape of the target tissue imposed onto the isocenter with the positioning points. After the particle beam starts scanning in a controlled path initiating from the isocenter, the positioning points can be used as the calibration for beam delivery after and before each round of scanning.

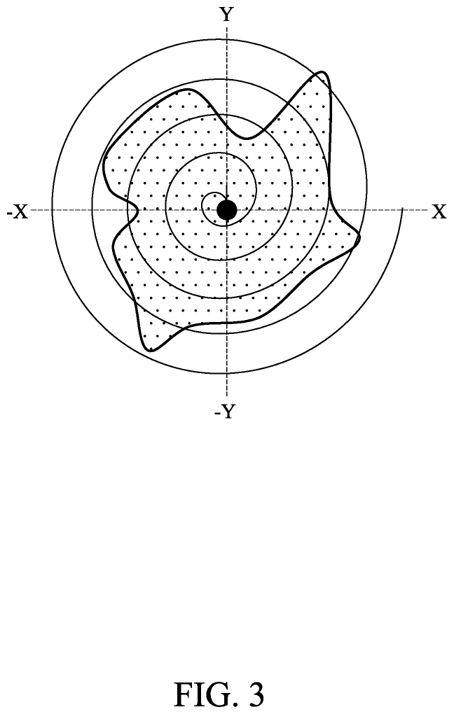

In the present disclosure, the method includes irradiating the target tissues with the particle beams scanning the target tissues initiating from the isocenter and following a controlled path. In an example, the controlled path follows a spiral pattern initiating from the isocenter, where the particle beams scan from the center of the target tissue outward toward the boundary of the target tissue (FIG. 3). During the scanning, the particle beam is under real-time monitoring and continuous control so that when the particle beam hits a region shown as normal cells, which could be predicted by comparing the scanning path with the image analysis done before the scanning, the particle beam would be deflected so that the energy of the beam does not deliver to the normal cells. In a spiral scanning pattern, the scanning is efficient because the particle beam can resume its original path faster after deflecting the beam to skip the normal cells in the scanning path. This is because the beam position is controlled by the magnets that move the beam in both the X and Y directions simultaneously in a spiral scanning pattern. By contrast, in the conventional scanning pattern such as the linear and zig-zag patterns, the magnets only move the beam along either X or Y direction; that is, when the beam moves along the X axis, the Y axis remains the same, and when the beam moves along the Y axis, the X axis remains the same.

Since it simply involves motion in only one direction (either X-axis or Y-axis) in a zig-zag scan, it takes more time to scan a defined target tissue to treat cancer. Also, it would be more prone to irradiate and damage normal cells. As such, the effective treatment area using a zig-zag scan is smaller than a spiral scan.

In the present disclosure, there provides a simultaneous motion in both the X- and Y-axes in the spiral scan. A spiral scan path is a scanning method that treats a target tissue with higher efficiency and higher protection. For example, the total length of the spiral scan path may be reduced with more focus on the central region of the tumorous mass as compared with the peripheral region thereof.

The controlled path of the beam scanning is defined by three coordinates according to an X, Y and Z direction; the Z coordinate corresponds to the beam direction, while the X and Y coordinates correspond to directions perpendicular to the Z direction. The particle beam irradiates the target tissue by scanning under a controlled path that forms a spiral pattern on the two-dimensional plane defined by the X and Y coordinates. Therefore, the target tissues consist of numerous layers, namely the 2-dimensional plane defined by the X and Y coordinates.

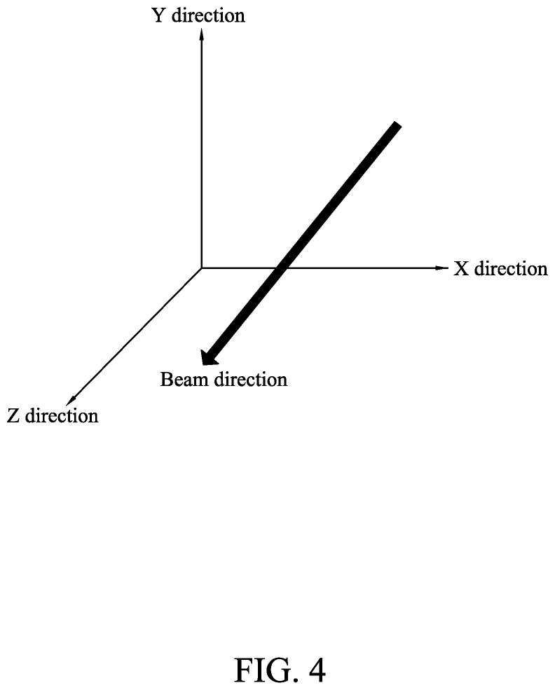

In the present disclosure, the particle beam irradiates the target tissue with one layer of the target tissue at a time with a fixed Z coordinate, starting from the isocenter of the layer, and repeats the scanning at another layer with a different fixed Z coordinate. Among different layers, the scanning always starts from the isocenter as the initiation point at each layer. To scan a different layer of the target tissue means scanning the target tissue at a different depth with a different Z coordinate, which is done by modulating the energy of the particle beam. A shown in FIG. 4, the Z coordinate represents the direction along which the beam is delivered into the target tissue, and when the energy of the beam increases, the beam is delivered deeper into the tissue.

In some embodiments of this disclosure, the method further improves the effectiveness of the treatment of the target tissue by the particle beam by introducing a reactant that concentrates preferentially in the cancer cells into the target tissue. Therefore, the method of the present disclosure involves treating the target tissue with a proton beam with the target tissue containing a reactant. The reactant can be a mixture of more than one composite; one of the composite is ⁷Li or ⁹Be to react with the proton from the proton beam and then release neutrons inside the target tissue, and another composite contains ¹⁰B to react with the neutrons and release α particles or γ rays that possess the power to damage the cells of the target tissue. For example, the composite containing ¹⁰B is one of the following: (L)-4-dihydroxy-borylphenylalanine (BPA), sodium mercaptoun decahydro-closo-dodecaborate (BSH), carbohydrate derivatives of BSH, a sodium salt of closo-B₁₀H₁₀²⁻ (GB-10), β-5-o-carboranyl-2V-deoxyuridine (D-CDU), 3-(dihydroxypropyl-carboranyl-pentyl) thymidine derivative (N5-20H), boron-containing porphyrins (H₂DCP), dequalinium derivatives (DEQ-B), derivatives of trimethoxyindoles, aziridines, derivatives of acridines, phenanthridines, carboranyl polyamines, Pt(II)-amine complexes, dibenzimidazoles, tribenzimidazoles, glucose molecules, mannose molecules, ribose molecules, glucose molecules, fucose molecules, galactose molecules, maltose molecules, lactose molecules, phosphates, phosphonates, phenylureas, thioureas, nitroimidazoles, amines, benzamides, isocyanates, nicotinamides, or azulenes.

In some embodiments, the method of the present disclosure involves treating the target tissue with a proton beam that irradiates the target tissue, where the target tissue is introduced with a boron-10 (¹⁰B) containing reactant including but not being limited to BPA, BSH, carbohydrate derivatives of BSH, GB-10, D-CDU, N5-2OH, H2DCP, DEQ-B, derivatives of trimethoxyindoles, aziridines, derivatives of acridines, phenanthridines, carboranyl polyamines, Pt(II)-amine complexes, dibenzimidazoles, tribenzimidazoles, glucose molecules, mannose molecules, ribose molecules, fucose molecules, galactose molecules, maltose molecules, lactose molecules, phosphates, phosphonates, phenylureas, thioureas, nitroimidazoles, amines, benzamides, isocyanates, nicotinamides, or azulenes. The target tissues introduced with the boron-10 (¹⁰B) containing reactant are irradiated with a proton beam, and the reactant inside the target tissue absorbs protons and then releases α particles or γ rays to damage cells of the target tissues. The reaction is represented by Formula 3 below:

p+¹¹B(¹⁰B)→nα or γ Formula 3.

In at least one embodiment of the present disclosure, a treatment planning system (TPS) is adopted with the particle therapy before treatment, during treatment and after treatment. Before treatment, the image of the target tissue to be treated by the particle beams is stored and analyzed in the TPS. The dimension information of the target tissue to be irradiated, the isocenter, the positioning points and the scan pattern path are subsequently determined by and stored in the TPS after the image analysis of the target tissue. During treatment, all the parameters adopted in delivering the particle beam such as the treatment time, the amount of the boron-containing reactant used, and the energy and intensity of the particle beam applied are all recorded in the TPS. The medical professionals could retrieve and utilize the information in the TPS for subsequent treatments, and thus TPS provides an efficient management system for implementing the particle therapy, which is often carried out several times in a treatment plan. The TPS comprises computer and data storage devices that store the information for use in scanning the target tissue. The stored information can be retrieved for subsequent treatments. The information may be transferred to the TPS via Internet or a cloud storage system by means of signal transmission.

The present disclosure has been described with embodiments thereof, and it is understood that various modifications, without departing from the scope of the present disclosure, are in accordance with the embodiments of the present disclosure. Hence, the embodiments described are intended to cover the modifications within the scope of the present disclosure, rather than to limit the present disclosure. The scope of the claims therefore should be accorded the broadest interpretation so as to encompass all such modifications.

What is claimed is:

1. A method for treating a target tissue in a subject in need thereof, the method comprising:
determining an isocenter of the target tissue; and
irradiating the target tissue with the isocenter as an initiation point by using a particle beam scanning under a controlled path.

2. The method according to claim 1, wherein the isocenter of the target tissue is determined with image analysis of a dimension and a location of the target tissue.

3. The method according to claim 2, wherein the image analysis is carried out with computed tomography (CT) scan, cone beam computed tomography (CBCT) scan, in-room CT scan, magnetic resonance imaging (MRI) scan, digital X-ray, an orthogonal X-ray, positron emission tomography (PET) scan, or any combination thereof.

4. The method according to claim 1, wherein the particle beam is a proton beam, a neutron beam, a carbon ion beam or any combination thereof.

5. The method according to claim 1, wherein the particle beam irradiates the target tissue in a spot scanning manner, a uniform scanning manner, a fast scanning manner, a scatter manner, or any combination thereof.

6. The method according to claim 1, wherein the target tissue is defined by X, Y and Z coordinates according to X, Y and Z directions, respectively, and wherein the Z direction corresponds to a direction of the particle beam, and the X direction and the Y direction are perpendicular to the Z direction.

7. The method according to claim 6, wherein the controlled path forms a spiral pattern on a two-dimensional plane defined by the X coordinate and the Y coordinate with a first fixed Z coordinate.

8. The method according to claim 7, further comprising, after completing scanning in the controlled path on the two-dimensional plane of the target tissue at the first fixed Z coordinate, irradiating the target tissue with the isocenter as an initiation point at a second fixed Z coordinate.

9. The method according to claim 8, further comprising, before initiating scanning the two-dimensional plane of the target tissue at the second fixed coordinate, calibrating the isocenter of the target tissue at the second fixed Z coordinate with one or more positioning points prescribed around the isocenter.

10. The method according to claim 9, wherein lines connecting the positioning points and crossing to each other defines the isocenter for calibration.

11. The method according to claim 6, wherein the Z coordinate of the controlled path is controlled by modulating energy of the particle beam.

12. The method according to claim 5, further comprising administering to the subject a first reactant comprising at least one of ¹⁰B and ¹¹B for reacting with at least one proton from the particle beam to release at least one of α particle and γ ray in the target tissue.

13. The method according to claim 12, wherein the first reactant is selected from the group consisting of (L)-4-dihydroxy-borylphenylalanine (BPA), sodium mercaptoun decahydro-closo-dodecaborate (BSH), a carbohydrate derivative of BSH, a sodium salt of closo-B₁₀H₁₀²⁻ (GB-10), β-5-o-carboranyl-2V-deoxyuridine (D-CDU), 3-(dihydroxypropyl-carboranyl-pentyl) thymidine derivative (N5-20H), boron-containing porphyrins (H₂DCP), dequalinium derivative (DEQ-B), a derivative of trimethoxyindole, aziridine, a derivative of acridine, phenanthridine, carboranyl polyamine, a Pt(II)-amine complex, dibenzimidazole, tribenzimidazole, a glucose molecule, a mannose molecule, a ribose molecule, a fucose molecule, a galactose molecule, a maltose molecule, a lactose molecule, phosphate, phosphonate, phenylurea, thiourea, nitroimidazole, amine, benzamide, isocyanate, nicotinamide, and azulene.

14. The method according to claim 5, further comprising administering to the subject a second reactant comprising a first composite for reacting with at least one proton from the particle beam and releasing at least one neutron in the target tissue, and a second composite for reacting with the at least one neutron and releasing at least one of α particle and γ ray in the target tissue.

15. The method according to claim 14, wherein the first composite contains one selected from the group consisting of ⁷Li, ⁹Be and any combination thereof.

16. The method according to claim 14, wherein the second composite contains ¹⁰B.

17. A method of treating cancer in a subject in need thereof, the method comprising implementing the method of claim 1 to the subject.

18. The method of claim 17, further comprising administering at least one additional therapy to the subject.

19. The method of claim 18, wherein the at least one additional therapy is chemotherapy, immunotherapy, surgery, radiotherapy, biotherapy, or any combination thereof.

20. The method of claim 19, wherein the immunotherapy triggers death of cancer cells induced by CD8⁺ T cells.
